(19) United States
(12) Patent Application Publication
(10) Pub. No.: US 2023/0174092 A1
(43) Pub. Date: Jun. 8, 2023

(54) VEHICLE AND METHOD OF DISPLAYING NITROGEN OXIDE EMISSIONS OF VEHICLE

(71) Applicants: HYUNDAI MOTOR COMPANY, SEOUL (KR); KIA CORPORATION, SEOUL (KR)

(72) Inventor: Bon Chang Koo, Seongnam-si (KR)

(21) Appl. No.: 17/990,180

(22) Filed: Nov. 18, 2022

(30) Foreign Application Priority Data
Dec. 7, 2021 (KR) 10-2021-0173516

(51) Int. Cl. B60W 50/14; B60K 35/00; G01N 33/00

(52) U.S. Cl. CPC B60W 50/14; B60K 35/00; G01N 33/0067; G01N 33/0037; B60W 2050/146; B60K 2370/16; B60K 2370/188

(57) ABSTRACT

A vehicle and a method of displaying nitrogen oxide emissions of the vehicle to accurately detect and easily display an amount of nitrogen oxide emitted from the vehicle includes receiving nitrogen oxide concentration values of exhaust gas in the vehicle, calculating an average value of the nitrogen oxide concentration based on the nitrogen oxide concentration values, calculating an amount of nitrogen oxide in the exhaust gas based on the average value of the nitrogen oxide concentration, calculating a nitrogen oxide index of the exhaust gas based on the amount of the nitrogen oxide, and displaying the calculated nitrogen oxide index for each of a plurality of predetermined sections through a display unit of a cluster of the vehicle.

### CROSS REFERENCE TO RELATED APPLICATIONS

This application is based on and claims priority under 35 U.S.C. § 119 to Korean Patent Application No. 10-2021-0173516, filed on Dec. 7, 2021, in the Korean Intellectual Property Office, the disclosure of which is incorporated by reference herein in its entirety.

### BACKGROUND

### 1. Field

The disclosure relates to a vehicle, and more particularly, to exhaust gas management of a vehicle.

### 2. Description of the Related Art

In order to remove nitrogen oxide and particulate matter emitted from a diesel engine, a selective catalytic reduction (SCR) and a diesel particulate filter (DPF) are applied.

The selective catalytic reduction occludes a reducing agent contained in exhaust gas, and removes the reducing agent through a reduction reaction with nitrogen oxide contained in the exhaust gas.

In order to precisely control the nitrogen oxide contained in the exhaust gas, it is necessary to accurately detect the nitrogen oxide contained in the exhaust gas.

### SUMMARY

It is an aspect of the disclosure to provide a vehicle capable of accurately detecting and easily displaying an amount of nitrogen oxide emitted from the vehicle.

Additional aspects of the disclosure will be set forth in part in the description which follows and, in part, will be obvious from the description, or may be learned by practice of the disclosure.

In accordance with an aspect of the disclosure, a method of displaying nitrogen oxide emissions of a vehicle includes receiving nitrogen oxide concentration values of exhaust gas in the vehicle, calculating an average value of the nitrogen oxide concentration based on the nitrogen oxide concentration values, calculating an amount of nitrogen oxide in the exhaust gas based on the average value of the nitrogen oxide concentration, calculating a nitrogen oxide index of the exhaust gas based on the amount of the nitrogen oxide, and displaying the calculated nitrogen oxide index for each of a plurality of predetermined sections through a display unit of a cluster of the vehicle.

The display method may further include receiving the nitrogen oxide concentration values for a plurality of preset periods, wherein the average value of the nitrogen oxide concentration may be an average value of the nitrogen oxide concentration for each period.

The nitrogen oxide index may be calculated through the following equation;

NOx index=(NOx amount)/(NOx allowance),

where the nitrogen oxide (NOx) allowance is an amount corresponding to a preset allowance reference value.

The nitrogen oxide (NOx) amount may be calculated through the following equation;

NOx amount=(0.001×46)/(3600×22.4)×(1/ρ)×(NOx concentration)×(exhaust gas flow rate),

where (0.001×46)/(3600×22.4)×(1/ρ) is a density of the exhaust gas emitted from the vehicle.

The exhaust gas flow rate is calculated through the following equation;

exhaust gas flow rate=(engine fuel consumption)×(engine rpm)^{(engine intake air volume)}.

The display method may further include receiving information on fuel consumption, revolutions per minute, and intake air volume of an engine.

A display of the nitrogen oxide index may be provided such that a plurality of sections including a section below the nitrogen oxide allowance and a section exceeding the nitrogen oxide allowance is divided and different preset colors are displayed depending on which section the nitrogen oxide index belongs to among the plurality of sections.

The plurality of sections may include a section less than or equal to 50% of the nitrogen oxide allowance, a section exceeding 50% and less than or equal to 100% of the nitrogen oxide allowance, and a section exceeding 100% of the nitrogen oxide allowance.

In accordance with another aspect of the disclosure, a vehicle includes a display unit of a cluster of the vehicle, a nitrogen oxide sensor provided to detect a nitrogen oxide concentration of exhaust gas of the vehicle, and a controller provided to receive nitrogen oxide concentration values from the nitrogen oxide sensor, calculate an average value of the nitrogen oxide concentration based on the nitrogen oxide concentration values, calculate an amount of nitrogen oxide in the exhaust gas based on the average value of the nitrogen oxide concentration, calculate a nitrogen oxide index of the exhaust gas based on the amount of the nitrogen oxide, and display the calculated nitrogen oxide index for each of a plurality of predetermined sections through the display unit of the cluster.

The controller may be provided to receive the nitrogen oxide concentration values for a plurality of preset periods, and the average value of the nitrogen oxide concentration may be an average value of the nitrogen oxide concentration for each period.

The controller may be provided to calculate the nitrogen oxide index through the following equation;

NOx index=(NOx amount)/(NOx allowance),

where the nitrogen oxide (NOx) allowance is an amount corresponding to a preset allowance reference value.

The controller may be provided to calculate the nitrogen oxide (NOx) amount through the following equation;

NOx amount=(0.001×46)/(3600×22.4)×(1/ρ)×(NOx concentration)×(exhaust gas flow rate),

where (0.001×46)/(3600×22.4)×(1/ρ) is a density of the exhaust gas emitted from the vehicle.

The controller may be provided to calculate the exhaust gas flow rate through the following equation;

exhaust gas flow rate=(engine fuel consumption)×(engine rpm)^{(engine intake air volume)}.

The controller may be provided to receive information on fuel consumption, revolutions per minute, and intake air volume of an engine.

A display of the nitrogen oxide index may be provided such that a plurality of sections including a section below the nitrogen oxide allowance and a section exceeding the nitrogen oxide allowance is divided and different preset colors are displayed depending on which section the nitrogen oxide index belongs to among the plurality of sections.

The plurality of sections may include a section less than or equal to 50% of the nitrogen oxide allowance, a section exceeding 50% and less than or equal to 100% of the nitrogen oxide allowance, and a section exceeding 100% of the nitrogen oxide allowance.

In accordance with another aspect of the disclosure, a method of displaying nitrogen oxide emissions of a vehicle includes receiving information on intake air volume, fuel consumption, and revolutions per minute of an engine, receiving nitrogen oxide concentration values of exhaust gas from a nitrogen oxide sensor for a plurality of preset periods, calculating a flow rate of exhaust gas discharged from the engine, calculating an average value of the nitrogen oxide concentration for each period based on the nitrogen oxide concentration values and the flow rate of the exhaust gas, calculating an amount of nitrogen oxide in the exhaust gas based on the average value of the nitrogen oxide concentration for each period, calculating a nitrogen oxide index of the exhaust gas based on the amount of nitrogen oxide, and displaying the calculated nitrogen oxide index through a display unit of a cluster of the vehicle, wherein a display of the nitrogen oxide index is provided such that a plurality of preset sections including a section below the nitrogen oxide allowance and a section exceeding the nitrogen oxide allowance is divided and different preset colors are displayed on the display unit depending on which section the nitrogen oxide index belongs to among the plurality of preset sections.

### BRIEF DESCRIPTION OF THE FIGURES

These and/or other aspects of the disclosure will become apparent and more readily appreciated from the following description of the embodiment, taken in conjunction with the accompanying drawings of which:

FIG. 1 is a diagram illustrating a configuration of an exhaust system of a vehicle according to an embodiment of the disclosure; and

FIG. 2 is a diagram illustrating a method of displaying nitrogen oxide emissions of the vehicle according to an embodiment of the disclosure.

### DETAILED DESCRIPTION

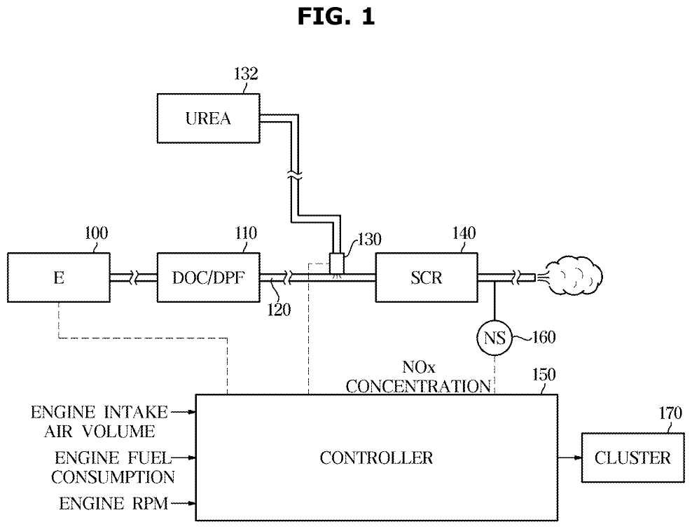

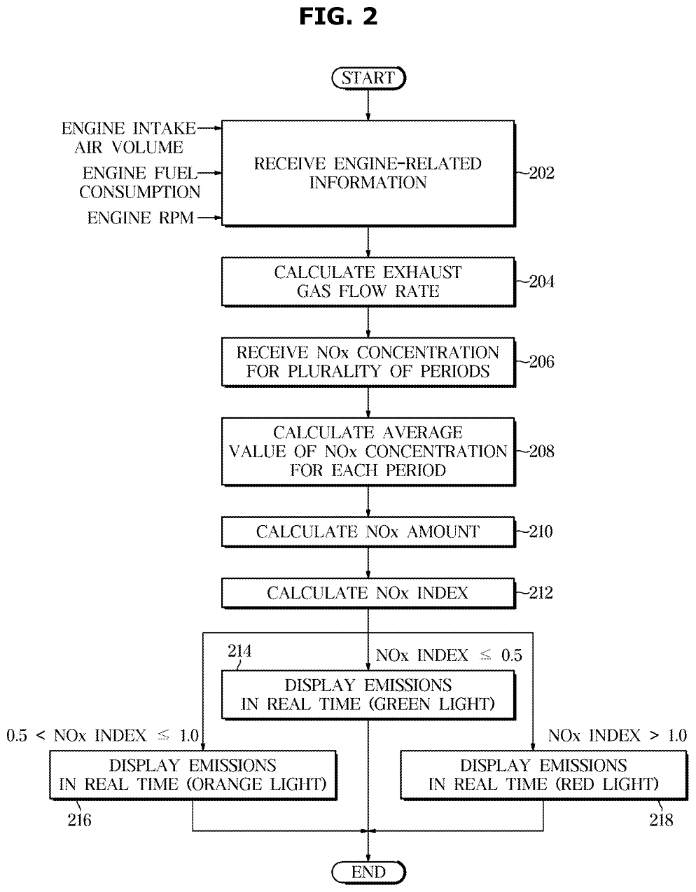

Hereinafter, a preferred embodiment of the disclosure will be described with reference to FIGS. 1 and 2.

FIG. 1 is a diagram illustrating a configuration of an exhaust system of a vehicle according to an embodiment of the disclosure.

As illustrated in FIG. 1, an exhaust system of a vehicle according to an embodiment of the disclosure includes an engine 100, an exhaust line 120, a catalyst unit (DOC/DPF) 110, a dosing unit 130, a selective catalytic reduction (SCR) 140, a nitrogen oxide sensor 160, and a controller 150.

Exhaust gas generated from the engine 100 is discharged to the outside of the vehicle through the exhaust line 120. The catalyst unit 110, the dosing unit 130, the selective catalytic reduction 140, and the nitrogen oxide sensor 160 are disposed in order in a middle portion of the exhaust line 120.

The catalyst unit 110 may be composed of a diesel oxidation catalyst (DOC) and a diesel particulate filter (DPF), and in general, the diesel oxidation catalyst (DOC) and the diesel particulate filter (DPF) are sequentially disposed. The catalyst unit 110 removes carbon monoxide and hydrocarbons contained in the exhaust gas, and a part of nitrogen dioxide (NO₂) is consumed by reacting with particulate matter collected in the diesel particulate filter (DPF).

The dosing unit 130 injects a reducing agent (e.g., urea) into the exhaust line 120 between the catalyst unit 110 and the selective catalytic reduction 140 through an injection tip. The reducing agent injected from the dosing unit 130 and nitrogen oxide (NOx) of the exhaust gas react in the selective catalytic reduction 140, and an amount of the nitrogen oxide (NOx) is reduced by this reaction. In FIG. 1, reference numeral 132 is a reducing agent storage unit 132 for storing the reducing agent for catalytic reduction.

The nitrogen oxide sensor 160 is provided to detect a concentration of the nitrogen oxide (NOx) contained in the exhaust gas. A concentration detection result of the nitrogen oxide sensor 160 is transmitted to the controller 150. The nitrogen oxide, which is a generic term for nitrogen oxide such as NO, N₂O, NO₂, N₂O₃, N₂O₅, refers to a mixture of NO and NO₂ when a subject is air pollutants and is denoted as ‘NOx’.

Engine-related information, such as intake air volume, fuel consumption, and revolutions per minute (RPM) of the engine 100 is input to the controller 150. In addition, information on the concentration of the nitrogen oxide (NOx) in the exhaust gas detected by the nitrogen oxide sensor 160 is input to the controller 150. The controller 150 calculates an average concentration of the nitrogen oxide (NOx), the amount of the nitrogen oxide (NOx), and an index of the nitrogen oxide (NOx) by period using information on the intake air volume, the fuel consumption, and the revolutions per minute (RPM) of the engine 100, and lights a cluster 170, which is a display unit, in a plurality of preset different colors depending on the calculated index of the nitrogen oxide (NOx).

FIG. 2 is a diagram illustrating a method of displaying nitrogen oxide emissions of the vehicle according to an embodiment of the disclosure.

As illustrated in FIG. 2, the controller 150 receives the engine-related information at 202. The engine-related information may include the intake air volume, the fuel consumption, and the revolutions per minute (RPM) of the engine 100.

The controller 150 calculates an exhaust gas flow rate of the vehicle using the received intake air volume, fuel consumption, and revolutions per minute (RPM) of the engine 100 at 204. The exhaust gas flow rate of the vehicle may be calculated through Equation 1 below.

Exhaust gas flow rate=(engine fuel consumption)×(engine rpm)^{(engine intake air volume)} Equation 1:

The controller 150 receives the nitrogen oxide (NOx) concentration information for a plurality of preset periods from the nitrogen oxide sensor 160 at 206. The plurality of preset periods may be 30 seconds, 60 seconds, and 120 seconds, respectively.

Also, the controller 150 calculates an average value of the nitrogen oxide (NOx) concentration for each of the plurality of preset periods at 208. That is, the controller 150 calculates the average value of the nitrogen oxide (NOx) concentration detected for 30 seconds, and then calculates the average value of the nitrogen oxide (NOx) concentration detected for 60 seconds, and then calculates the average value of the nitrogen oxide (NOx) concentration detected for 120 seconds. Calculating the average value for the plurality of preset periods as described above is to reduce a deviation of the nitrogen oxide sensor 160. That is, various models of different manufacturers may be used as the nitrogen oxide sensor 160. The nitrogen oxide sensor 160 applies an average value for a different period for each manufacturer or model. In an embodiment of the disclosure, by calculating average values for periods of 30 seconds, 60 seconds, and 120 seconds, a period suitable for the actually adopted nitrogen oxide sensor 160 may be applied.

The calculation of the average value as described above is to improve a measurement error of the nitrogen oxide sensor 160. When a portable exhaust gas measurement device (aka PEMS) is used, a composition of exhaust gas emitted from a vehicle may be measured very accurately. However, because the portable exhaust gas measurement device is expensive, it is a burdensome device for an individual driver to purchase the portable exhaust gas measurement device. On the other hand, the nitrogen oxide sensor 160 is cheaper than the portable exhaust gas measurement device, but a measurement error may occur. Therefore, it is preferable to use the relatively inexpensive nitrogen oxide sensor 160 and to reduce an error of the nitrogen oxide sensor 160 by calculating an average value for every predetermined period.

When the exhaust gas flow rate of the vehicle and the nitrogen oxide (NOx) concentration are calculated, the controller 150 calculates the amount of nitrogen oxide (NOx) based on the exhaust gas flow rate and the nitrogen oxide (NOx) concentration at 210. The amount of the nitrogen oxide (NOx) may be calculated through Equation 2 below.

NOx amount=(0.001×46)/(3600×22.4)×(1/ρ) (NOx concentration)×(exhaust gas flow rate) Equation 2

In Equation 2, (0.001×46)/(3600×22.4)×(1/ρ) is a density of exhaust gas, and 0.001 and 3600 are for converting kilograms (kg) and hours to grams and seconds, respectively,

Next, the controller 150 calculates the nitrogen oxide (NOx) index based on the amount of the nitrogen oxide (NOx) calculated above at 212. The nitrogen oxide (NOx) index may be calculated through Equation 3 below.

NOx index=(NOx amount)/(NOx allowance) Equation 3:

In Equation 3, the amount of the nitrogen oxide (NOx) is a value calculated in step 210 above, and the nitrogen oxide (NOx) allowance is a standard value allowed by each national standard or international standard. For example, when the nitrogen oxide emission standard of ‘Euro 7’ is 30 mg/km, the nitrogen oxide (NOx) allowance in Equation 3 may be ‘30’. That is, the nitrogen oxide (NOx) index of the disclosure indicates what percentage a current amount of the nitrogen oxide (NOx) in the vehicle compared to the nitrogen oxide allowance corresponds to.

When the calculated nitrogen oxide (NOx) index is less than or equal to 50% of the allowance (NOx index≤0.5), the controller 150 displays in real time that the nitrogen oxide (NOx) emissions of the vehicle are 50% or less of the allowance by lighting the cluster 170, which is the display unit, in green at 214.

When the calculated nitrogen oxide (NOx) index exceeds 50% of the allowance but is less than or equal to 100% (0.5<NOx index≤1.0), the controller 150 displays in real time that the nitrogen oxide (NOx) emissions of the vehicle do not exceed the allowance by lighting the cluster 170, which is the display unit, in orange at 216.

When the calculated nitrogen oxide (NOx) index exceeds 100% of the allowance (NOx index>1.0), the controller 150 displays in real time that the nitrogen oxide (NOx) emissions of the vehicle exceed 100% of the allowance by lighting the cluster 170, which is the display unit, in red at 218.

Lighting in different colors to display the nitrogen oxide (NOx) index is not limited to the colors of green, orange, and red, and a plurality of more sections including a section below the allowance and a section exceeding the allowance may be divided and may be displayed in more different colors. In addition, the nitrogen oxide (NOx) index may be displayed on a display of a vehicle multimedia device in addition to the cluster 170,

As is apparent from the above, the disclosure can accurately detect and easily display an amount of nitrogen oxide emitted from a vehicle.

The embodiment disclosed above is merely illustrative of the technical idea, and various modifications, changes and substitutions may be made by those skilled in the art without departing from the essential characteristics. Therefore, the embodiment and the accompanying drawings are for explanation rather than limiting the technical idea, and the scope of the technical idea is not limited by this embodiment and the accompanying drawings. The scope of protection should be interpreted by the following claims, and all technical ideas within the scope equivalent thereto should be construed as being included in the scope of rights.

1. A method of displaying nitrogen oxide emissions of a vehicle comprising:
receiving, by a controller, nitrogen oxide concentration values of exhaust gas in the vehicle;
calculating an average value of the nitrogen oxide concentration based on the nitrogen oxide concentration values;
calculating an amount of nitrogen oxide in the exhaust gas based on the average value of the nitrogen oxide concentration;
calculating a nitrogen oxide index of the exhaust gas based on the amount of the nitrogen oxide; and
displaying the calculated nitrogen oxide index for each of a plurality of predetermined sections through a display unit of a cluster of the vehicle.

2. The display method according to claim 1, further comprising:
receiving the nitrogen oxide concentration values for a plurality of preset periods,
wherein the average value of the nitrogen oxide concentration is an average value of the nitrogen oxide concentration for each period of the preset periods.

3. The display method according to claim 1, wherein the nitrogen oxide index is calculated through the equation:
NOx index=(NOx amount)/(NOx allowance),
where the nitrogen oxide (NOx) allowance is an amount corresponding to a preset allowance reference value.

4. The display method according to claim 3, wherein the nitrogen oxide (NOx) amount is calculated through the equation:
NOx amount=(0.001×46)/(3600×22.4)×(1/ρ)×(NOx concentration)×(exhaust gas flow rate).
where (0.001×46)/(3600×22.4)×(1/ρ) is a density of the exhaust gas emitted from the vehicle.

5. The display method according to claim 4, wherein the exhaust gas flow rate is calculated through the equation:
exhaust gas flow rate=(engine fuel consumption)×(engine rpm)^{(engine intake air volume)}.

6. The display method according to claim 5, further comprising receiving information on fuel consumption, revolutions per minute, and intake air volume of an engine.

7. The display method according to claim 3, wherein a display of the nitrogen oxide index is provided such that a plurality of sections including a section below the nitrogen oxide allowance and a section exceeding the nitrogen oxide allowance is divided, and different preset colors are displayed depending on which section the nitrogen oxide index belongs to among the plurality of sections.

8. The display method according to claim 7, wherein the plurality of sections includes a section less than or equal to 50% of the nitrogen oxide allowance, a section exceeding 50% and less than or equal to 100% of the nitrogen oxide allowance, and a section exceeding 100% of the nitrogen oxide allowance.

9. A vehicle comprising:
a display unit of a cluster of the vehicle;
a nitrogen oxide sensor configured to detect a nitrogen oxide concentration of exhaust gas of the vehicle; and
a controller configured to receive nitrogen oxide concentration values from the nitrogen oxide sensor, calculate an average value of the nitrogen oxide concentration based on the nitrogen oxide concentration values, calculate an amount of nitrogen oxide in the exhaust gas based on the average value of the nitrogen oxide concentration, calculate a nitrogen oxide index of the exhaust gas based on the amount of the nitrogen oxide, and display the calculated nitrogen oxide index for each of a plurality of predetermined sections through the display unit of the cluster.

10. The vehicle according to claim 9, wherein the controller is configured to receive the nitrogen oxide concentration values for a plurality of preset periods, and
the average value of the nitrogen oxide concentration is an average value of the nitrogen oxide concentration for each period.

11. The vehicle according to claim 9, wherein the controller is configured to calculate the nitrogen oxide index through the equation;
NOx index=(NOx amount)/(NOx allowance),
where the nitrogen oxide (NOx) allowance is an amount corresponding to a preset allowance reference value.

12. The vehicle according to claim 11, wherein the controller is configured to calculate the nitrogen oxide (NOx) amount through the equation;
NOx amount=(0.001×46)/(3600×22.4)×(1/ρ)×(NOx concentration)×(exhaust gas flow rate),
where (0.001×46)/(3600×22.4)×(1/ρ) is a density of the exhaust gas emitted from the vehicle.

13. The vehicle according to claim 12, wherein the controller is configured to calculate the exhaust gas flow rate through the equation;
exhaust gas flow rate=(engine fuel consumption)×(engine rpm)^{(engine intake air volume)}.

14. The vehicle according to claim 13, wherein the controller is configured to receive information on fuel consumption, revolutions per minute, and intake air volume of an engine.

15. The vehicle according to claim 11, wherein a display of the nitrogen oxide index is provided such that a plurality of sections including a section below the nitrogen oxide allowance and a section exceeding the nitrogen oxide allowance is divided, and different preset colors are displayed depending on which section the nitrogen oxide index belongs to among the plurality of sections.

16. The vehicle according to claim 15, wherein the plurality of sections includes a section less than or equal to 50% of the nitrogen oxide allowance, a section exceeding 50% and less than or equal to 100% of the nitrogen oxide allowance, and a section exceeding 100% of the nitrogen oxide allowance.

17. A method of displaying nitrogen oxide emissions of a vehicle comprising:
receiving, by a controller, information on intake air volume, fuel consumption, and revolutions per minute of an engine;
receiving nitrogen oxide concentration values of exhaust gas from a nitrogen oxide sensor for a plurality of preset periods;
calculating a flow rate of exhaust gas discharged from the engine;
calculating an average value of the nitrogen oxide concentration for each period based on the nitrogen oxide concentration values and the flow rate of the exhaust gas;
calculating an amount of nitrogen oxide in the exhaust gas based on the average value of the nitrogen oxide concentration for each period;
calculating a nitrogen oxide index of the exhaust gas based on the amount of nitrogen oxide; and
displaying the calculated nitrogen oxide index through a display unit of a cluster of the vehicle;
wherein a display of the nitrogen oxide index is provided such that a plurality of preset sections including a section below the nitrogen oxide allowance and a section exceeding the nitrogen oxide allowance is divided and different preset colors are displayed on the display unit depending on which section the nitrogen oxide index belongs to among the plurality of preset sections.
